(19) United States
(12) Patent Application Publication
(10) Pub. No.: US 2023/0014726 A1
(43) Pub. Date: Jan. 19, 2023

(54) INFRASTRUCTURE AND METHODOLOGY FOR PRODUCING CANNABIS

(71) Applicant: CICADA LIMITED, ST. CLEMENT (GB)

(72) Inventors: EVAN SMITH, ANCASTER (CA); GUILLAUME DUVEAU, ST. CLEMENT (GB); RENATA LEGIERSKA, LONDON (GB)

(21) Appl. No.: 17/785,047

(22) PCT Filed: Dec. 18, 2020
(86) PCT No.: PCT/CA2020/051756

§ 371 Date: Jun. 13, 2022

Related U.S. Application Data
(60) Provisional application No. 62/949,556, filed on Dec. 18, 2019.

(51) Int. Cl. A01G 22/00; E04H 5/08

(52) U.S. Cl. CPC A01G 22/00; E04H 5/08

(57) ABSTRACT

A production plan for producing cannabis in a facility comprising a building module comprising five growth chambers of substantially equal size and a GMP processing area comprises a system of concurrent cyclic processes (SCCPs) for carrying out cannabis processing steps of hardening; growth, wet hanging, in-process quality control, transfer to GMP drying; in-process quality control; trimming; curing, and grading/packing. The SCCPs is based upon a minimum cycle time (MCT) defined as MCT=FP/5, where FP is a flowering period for a selected cannabis species, and 5 is the number of growth chambers in which the cannabis species will be grown. A cannabis production facility comprises a building module having five growth chambers of substantially equal size, a GMP processing area and a production plan. A method is provided for designing a cannabis production facility for assessing the feasibility thereof at a concept stage.

### RELATED APPLICATIONS

This application claims the benefit of U.S. patent application Ser. No. 62/949,556 filed on Dec. 18, 2019, the entire teachings of which are incorporated herein by reference.

### FIELD OF THE INVENTION

The invention relates to facilities for the indoor production of high-quality cannabis to meet international Good Manufacturing Practice (GMP) for medicinal products, and to the design of large-scale batch manufacturing facilities, and specifically to the design of biopharmaceutical (herbal medicine) drug manufacturing processes.

### BACKGROUND

The regulatory standards for the production of medicinal products (cannabis or otherwise) are very stringent. In order to be licensed to produce a medicinal product, the manufacturing facility and all production practices must conform to the rigorous regulatory standards of the primary healthcare regulators and government agencies and licensing authorities. These include the European Medicines Agency and national competent authorities such as the Medicines and Healthcare products Regulatory Agency (MHRA) in the UK, the Federal Institute for Drugs and Medicinal Devices (BfArM) in Germany and the United States Food and Drug Administration (FDA). In order to obtain and maintain appropriate licences and certifications, the operators of a production facility must have procedures in place to validate that product quality and safety standards are being met at all times. The fundamental requirements of GMP in the manufacturing of medicines are the tracking and quality assurance of all components, product precursors and finished products and the prevention of mix-ups and cross-contamination, which are the main causes of product adulteration and recalls in the pharmaceutical industry.

The production of medicinal cannabis is subject to the highest level of regulatory control in all jurisdictions with legal cannabis programmes. However, it is also a young industry, with systems and processes that are constantly evolving. Aligning cannabis manufacturing processes with international efforts to harmonise the production of medicinal products—as set out in guidelines published by the International Conference on Harmonization (ICH), the International Society for Pharmaceutical Engineering (ISPE) or the American Society for Testing and Materials (ASTM)—provides the best means to stay at the forefront of regulatory requirements.

A conventional indoor cannabis growth/production facility is typically a warehouse-like space fitted with lighting, water and nutrient supply equipment. The facility is typically divided into individual rooms, each of which is maintained at a different lighting, nutrient and water delivery environment matched to the growth stages of the cannabis plants. Facilities typically have a cuttings/potting room, a vegetative growth room and one or more flowering rooms. Separate vegetation and flowering rooms are commonly used in the production of cannabis in large part due to different types of fixed spectrum lighting used for the vegetation and flowering growth phases. The plants are physically moved from one room to another as they progress through their growth stages. Unfortunately, moving the plants between rooms multiple times during their growth cycle leads to significant risk of cross contamination between plants, and contamination of the plants by persons tasked with inspecting, handling and moving the plants. In such circumstances, demonstrating that risks to the quality and safety of the products are adequately mitigated at each stage becomes an onerous compliance task.

There is a need for an innovative production facility and process designed for the production of high-quality cannabis which can be deployed anywhere in the world and is able to conform to multiple international regulatory standards by virtue of its validation-ready facility and process design, which meets international GMP standards for medicinal products.

The design, architecture and engineering of biopharmaceutical manufacturing facilities is a multi-billion-dollar industry because of the complex nature of biopharmaceutical production. The design of biopharmaceutical products, including medicinal cannabis, occurs in discrete phases. The first phase is the conceptual design phase that begins with the identification of the high-level steps of the process that will produce the desired biopharmaceutical product. After the high-level process steps have been identified, the unit operations associated with each of the high-level steps are identified. Unit operations are discrete process steps that make up the high-level process steps. The unit operation level production process is typically designed on a case-by-case basis and is prone to errors and inefficiencies. Scale calculations for each of the process steps/unit operations are performed to determine the size and capacity of the room or equipment necessary to produce the desired amount of product per batch. Since the scale calculations are developed from the original conceptual design parameters, they are also subject to the same potential errors inherent in the initial conceptual design base. Typically, a process flow diagram is generated after the scale calculations for the unit operations have been performed. The process flow diagram graphically illustrates the process equipment such as growing rooms and processing rooms necessary to accommodate the process for a given batch scale. A preliminary facility layout for the plant is developed from the process flow diagram and preliminary equipment list. The preliminary facility layout usually begins with a bubble or block diagram of the facility that illustrates the adjacencies of rooms housing different high-level steps, as well as a special arrangement which dimensions of the space and square footage of the building. From this information a preliminary equipment layout for the facility is prepared. The preliminary equipment layout attempts to show all the rooms in the facility, including corridors, staircases, etc. Next, mechanical, electrical, and plumbing engineers estimate the mechanical, electrical, and plumbing needs of the facility based on the facility design layout and the utility requirements of the manufacturing equipment. Since the preliminary facility layout is developed from the original conceptual design parameters, it can become potentially subject to the same errors inherent in the initial conceptual design base. The next phase is the detailed design of process piping, mechanical, electrical systems, plumbing systems, tanks, instrumentation, controls and hardware. Again, since the detailed design work is developed from the original design parameters, any errors inherent in the initial design phases may flow through into detailed designs. Reworking preliminary and detailed drawings and process diagrams due to errors in the conceptual design phase can cost thousands of dollars per diagram. The inability to accurately model and simulate the biopharmaceutical production process drives inaccurate initial design. Often, these inaccuracies result in changes to the design and construction diagrams or repair and reconstruction of the facility during the construction phase, resulting in millions of dollars in additional cost. There is a need for a system and method for accurately simulating and modelling a biopharmaceutical production process to allow designers to reduce design errors early on in the design process.

Indoor horticultural production is resource intensive in terms of water and electricity consumption. The cost of maintaining optimized growing environments in warehouse-sized facilities is considerable. There is a need for a facility which operates efficiently to eliminate any idle time, meaning that all growing areas are fully operational at all times in order to see an optimal product output from the resources consumed.

There is a need for a facility which can facilitate expansion and tech transfer whilst limiting the size of individual chambers and potential loss, designed so as to maximise available floorspace in an efficient manner for continuous supply.

There is a need for a production facility with digital and mechanical systems which can be calibrated to optimize environmental conditions to achieve the desired characteristics of the end products while employing physical and temporal separations to prevent cross-contamination.

### SUMMARY OF THE INVENTION

A production plan is provided for producing cannabis in a facility comprising a building module consisting of five growth chambers of substantially equal size and a GMP processing area. Each of the growth chambers is fully equipped to provide an optimal growing environment and has all the critical material attributes (CMAs) necessary to support the natural lifecycle of the cannabis plant. Each growth chamber is further equipped with a building management system (BMS) and Environmental Management System (EMS) comprising a system of sensors calibrated to monitor critical process steps as they relate to the CMAs and to support operations in the chamber. The production plan comprises a System of Concurrent Cyclic Processes (SCCPs) by which the following cannabis processing steps are carried out: hardening; growth, wet hanging, in-process quality control, transfer to GMP drying; in-process quality control; trimming; curing, and grading/packing. The SCCPs is based upon a Minimum Cycle Time (MCT), defined as MCT=FP/5, where FP is a flowering period for a selected cannabis species, and 5 is the number of growth chambers in which the cannabis species will be grown. The cycle length of the hardening step is 1 MCT. The cycle length of the growing step is 5 MCT. The cycle length of the wet hanging step is 1 MCT. The cycle length of the GMP drying step is 1 MCT. The cycle length of the curing step is at least 3 MCT. The grading/packing cycle is 1 MCT. The production plan further comprises a plurality of cleaning sub-cycles interleaved between each of the processing steps, and the cycle length of each of the cleaning sub-cycles is one day. The trimming step is performed in a common room and has a unit of operation based upon the performance of the trimming activity, such that each batch of cannabis is trimmed within a sub-cycle having a duration of 1 MCT divided by 5, where 5 is the number of growth chambers being operated.

A cannabis production facility comprises a building module having five growth chambers of substantially equal size. Each of the growth chambers is fully equipped to provide an optimal growing environment having all critical material attributes (CMAs) necessary to support the natural lifecycle of the cannabis plant. Each growth chamber is further equipped with a Building Management System (BMS) and Environmental Management System (EMS) comprising a system of sensors calibrated for monitoring critical process steps as they relate to the CMAs. The building module further comprises a support operations chamber having a gown-up area; a materials receiving area; a hardening room; a wet hanging room; and, a gown-down area. The cannabis production facility further comprises a GMP processing area dedicated to post-harvest processing. The GMP processing facility has a drying area; a quality control laboratory; a trimming room; a curing area; and a grading and packing area. A production plan, which is a system of concurrent cyclic processes (SCCPs) controls the following cannabis processing steps: hardening; growth, wet hanging, in-process quality control, transfer to GMP drying; in-process quality control; trimming; curing, and grading/packing.

A method is provided for designing a cannabis production facility for assessing the feasibility thereof at a concept stage. The proposed production facility would have a building module comprising five growth chambers of substantially equal size. Each growth chamber would be fully equipped to provide an optimal growing environment having all critical material attributes (CMAs) necessary to support the natural lifecycle of the cannabis plant, a Building Management System (BMS) and Environmental Management System (EMS) comprising a system of sensors calibrated for monitoring critical process steps as they relate to the critical material attributers (CMAs). A support operations chamber and a GMP processing area would also be part of the proposed facility. The first step in the method is providing a production plan comprising a system of concurrent cyclic processes (SCCPs) by which the following cannabis processing steps are carried out: hardening; growth, wet hanging, in-process quality control, transfer to GMP drying; in-process quality control; trimming; curing, and grading/packing. The next step is to determine a cannabis canopy size value per growth chamber and input the cannabis canopy size value into the production plan. Then, a flowering period value is determined for a selected cannabis species and inputting said cannabis canopy size value into the production plan. The next step is to determine a desired number of building modules each of which comprises five growth chambers, and input the desired number of building modules into the production plan. Then, a desired minimum curing duration is determined and the value is input into the production plan. The next step is determining a desired harvest window value and inputting the desired harvest window value into the production plan. Finally, a value is determined for the anticipated input costs needed for daily operations and the value is inputted into the production plan. When a model is generated with the above listed values inputted into the production plan, a user can make judgements regarding the feasibility of the proposed facility.

### BRIEF DESCRIPTION OF THE DRAWINGS

FIG. 1 is a schematic overview of the building module of a production facility for cannabis in accordance with the present invention.

FIG. 2 is a schematic overview of the pharmaceutical processing area of a production facility for cannabis in accordance with the present invention.

FIG. 3 is a Gantt chart showing the processing steps for one production cycle for one batch of plants according to the production plan.

FIG. 4 is a Gantt chart showing the processing steps for one batch of one production cycle for four batches of plants, occupying five growth chambers according to the production plan.

FIG. 5 is a Gantt chart showing the processing steps for the production cycle in cyclic steady state model according to the production plan.

FIG. 6 is a Gantt chart showing the post-harvest processing steps in cyclic steady state model according to the production plan.

FIG. 7 is a Gantt chart showing all cleaning steps in place for the production cycle and the post-harvest processing steps in cyclic steady state model according to the production plan.

### DETAILED DESCRIPTION

Certain terminology is used in the following description for convenience only and is not limiting. The words “lower,” “bottom,” “upper,” and “top” designate directions in the drawings to which reference is made. Unless specifically set forth herein, the terms “a,” “an” and “the” are not limited to one element, but instead should be read as meaning “at least one.” The terminology includes the words noted above, derivatives thereof and words of similar import.

In order to function effectively, a biopharmaceutical manufacturing facility must include sufficient space for at least the following areas: shipping and receiving area, sample testing and receiving, warehouse, warehouse staging area, manufacturing operations (including growing chambers), packaging operations, secure storage (vault), support areas (e.g., locker rooms, gowning rooms, changing rooms and amenities), and a quality control laboratory.

The focus of the present invention is on the manufacturing operations areas which, for the production of medicinal cannabis, comprise a growing and harvesting environment and a designated area for post harvest processing. A combination of physical and temporal separations within the overall manufacturing process ensures that there is no risk of product mix up or cross contamination. The design is based on the principles of quality by design, a philosophy that goes beyond merely ensuring regulatory compliance to embodying the ultimate goals behind the regulations: safeguarding consumer safety and product quality by virtue of validated processes and system controls. The production facility and the production plan integrate biological sciences, process engineering, quality assurance and international best practice standards to develop design solutions that guarantee product safety and achieve maximum quality and efficiency. All critical systems and processes embodied in the present invention have been designed in accordance with harmonized global GMP standards and can be readily validated, making the production facility and the production plan deployable anywhere in the world.

In accordance with the present invention, a production facility is operated in accordance with a production plan that schedules all activities which occur in the production facility. The digital architecture of the production plan integrates software and hardware in order to automate repetitive tasks, program all manufacturing cycles well into the future, track individual batches of cannabis throughout their production cycle, and use built-in sensors to gather data for the purposes of ongoing risk management, traceability and compliance, and future product improvement.

The objective of the production facility is to provide near-continuous manufacturing of small batch, high quality medicinal plants within a cGMP compliant space. The facility and process design aim for simultaneous conformance and performance through a combination of architecture and mathematics, with each contributing to the other to create a highly ordered process with a carefully timed sequence of downstream process steps/unit operations. A combination of physical and temporal separations within the overall manufacturing process eliminates the risk of product mix up or cross contamination. It is the production plan which enables the monitored and concurrent growing and processing of any variety of cannabis plants (or other botanical materials) within the same production facility at the same time, without the risk of mix-ups or cross-contamination.

The basic operating principle underlying the production plan is the accommodation of the flowering periods required for the growth and maturation of the two cannabis sub-species. The genetic makeup of the two main cannabis sub-species, Sativa and Indica, dictates their growing cycles (time to harvest). Sativas originate from equatorial regions with long daylight cycles and require approximately 85 days to flower. Indicas originate from the northern and southern hemispheres with less sunlight and require only approximately 65 days to flower. The production facility accommodates the flowering of any Sativa, and any Indica variety, and hybrids thereof. The basic unit of the production plan is a Minimum Cycle Time (MCT). It has been determined that the most efficient use of growing space is to establish five (5) growth chambers. In order to determine the length of an MCT for growing cannabis, the flowering periods of the two main species is notionally divided into five cycles: a 5×17-day Sativa cycle=85-day, and a 5×13-day Indica cycle=65-day. Thus, the MCT for a sativa cannabis crop is approximately 17 days, and for an indica cannabis crop the MCT is approximately 13 days. According to the production plan each, one of the growth chambers is planted sequentially, according to the MCT for the selected plant variety. In the examples and drawing figures provided in the present application, the minimum cycle time is taken to be 17 days. It should be understood that the production plan for indica or hybrid species will be analogous to the examples shown, but based upon minimum cycle times which will vary as a function of the flowering period the particular species. MCT could be defined as follows: MCT=FP/5, where FP is the flowering period for the species to be grown, and 5 is the number of growth chambers.

The production facility is “strain agnostic” and will accommodate the growing cycle of any indica or sativa variety by dividing their flowering periods by 5. While not all strains fit perfectly into a 65-day or 85-day production cycle, the production facility according to the present invention includes a “strain sieve” which is essentially a method for adjusting the production plan to marginally increase or decrease the minimum cycle time to accommodate the flowering period of any cannabis variety and to allow production cycles having different minimum cycle times to operate concurrently without any days or points in the cycle where a single room or resource is required to process plants in different cycles.

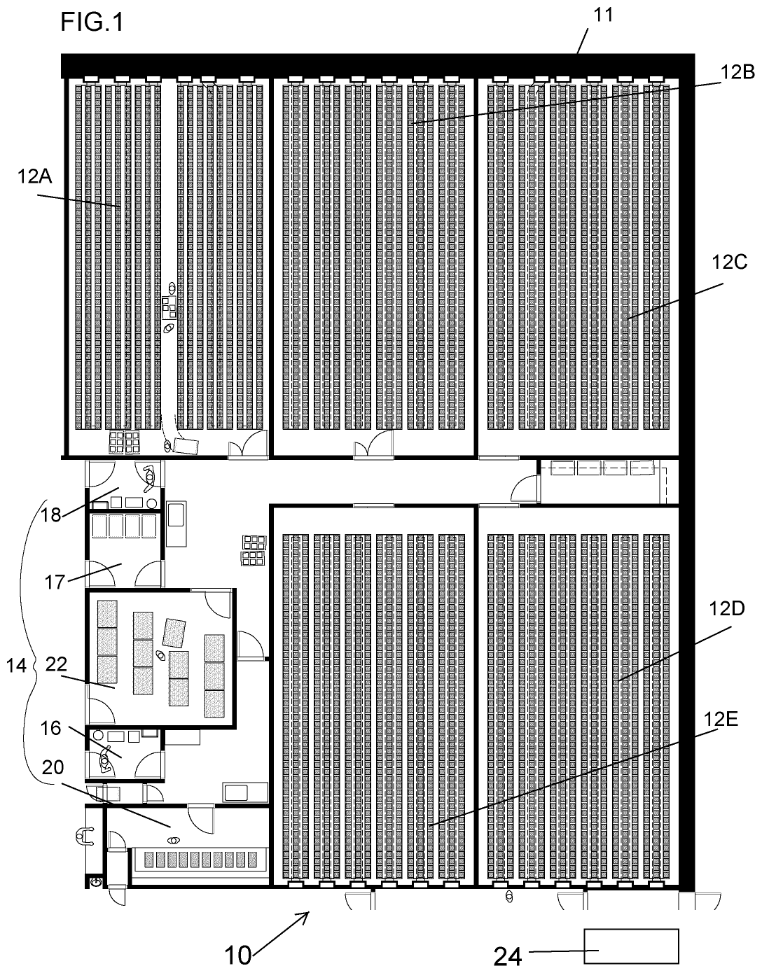

As can be seen in FIG. 1, a cannabis production facility according to the present invention is shown generally by reference numeral 10. The entire facility and all contained processes stem from the modular physical structure of the building module 11, comprising five (combined vegetation/flowering) growth chambers, arranged in a group with centralised access to maximise production space and minimise hallways/dead space. The layout could be described as a rectangle bisected along the narrow north-south dimension, then each half trisected along its narrow east-west dimension. The result is six equal rectangular areas, with five serving as growth chambers 12, and the sixth is the support operations chamber 14 dedicated to support activities in support of the growth chambers 12.

The support operations chamber 14 is provided with a gown-up room 16 which is maintained in a sterile state and contains a supply of sterile gowns etc. A gown down area 18 is provided so that workers can remove and dispose of contaminated garments. The support operations chamber 14 provides controlled access for materials and personnel including a gown-up area 16 where workers can put on sterile protective garments, a materials receiving area 17, a gown-down area 18 for removal of dirty garments, a cutting/clone hardening room 20 and a wet hanging room 22.

In keeping with the need to comply with requirements of GMP at all times, sterile conditions are maintained in each of the growth chambers, processing rooms and storage areas. All doors and hallways within the production facility are fitted with controlled access systems and air locks to ensure that all growth chambers and all processing rooms are separated from one another, and are closed off and cleaned between all process steps involving different batches of plant material. Likewise, all hallways and transportation pathways within the production facility can be closed off and cleaned before and after any plant material is transferred from one area of the production facility to another.

Each of the five growth chambers 12 (12A, 12B, 12C, 12D, 12E) is equal in size and fully equipped to provide an optimal growing environment which provides all of the critical material attributes (CMAs) to support the natural lifecycle of the cannabis plant. These include the HVAC equipment, lighting, and other equipment and infrastructure required for this purpose. Each of the five growth chambers is also equipped with a Building Management System (BMS) and an Environmental Management System (EMS), comprising an integrated system of sensors calibrated for monitoring the critical process steps as they relate to the CMAs.

The physical infrastructure and the digital infrastructure together accommodate the functionality of the internet of things, as devices with built-in sensors will capture data which can subsequently be used to detect patterns, make recommendations, and identify possible problems before they occur. This enables the integration of all equipment and systems that control the key variables that affect the quality of cannabis plants, including lighting spectrum and intensity, temperature, humidity, levels of oxygen and carbon dioxide, microbiome, irrigation, and nutrient regimen.

For greater specificity, Table 1 lists the Critical Material Attributes (CMAs) which are the environmental inputs according to the present invention which must be controlled within defined critical process parameters (CPPs) in the growth chambers 12, as they affect the Critical Quality Attributes (CQAs) of the cannabis products. The critical material attributes are assigned M-Numbers in the context of environmental controls defined for each process step so that they can be tracked and correlated against variations in the product quality (vs CQAs) in accordance with the production plan 13.

TABLE 1
CRITICAL MATERIAL ATTRIBUTES (CMAs)

| M-Number | Attribute |
|---|---|
| 1.0 | Rooting Compound |
| 1.1 | Indole-Butyric Acid (IBA) |
| 1.2 | Willow Water |
| 2.0 | Growing Medium |
| 2.1 | Material - Peat/Perlite |
| 2.2 | Size & Shape |
| 2.3 | PH Content |
| 2.4 | Microbiome Present |
| 2.4.1 | ~insert~ |
| 3.0 | Light |
| 3.1 | Intensity |
| 3.2 | Spectra |
| 3.2.1 | Far Red |
| 3.2.2 | Red |
| 3.2.3 | Yellow |
| 3.2.4 | Green |
| 3.2.5 | Blue |
| 3.2.6 | Ultra Violet |
| 3.3 | Photoperiod |
| 4.0 | Gasses |
| 4.1 | Particle Count |
| 4.2 | Content - Lights On |
| 4.2.1 | Oxygen (O) |
| 4.2.2 | Carbon Dioxide (CO2) |
| 4.2.3 | Nitrogen (N) |
| 4.2.4 | Ethylene (C2H4) |
| 4.3 | Content - Lights Off |
| 4.3.1 | Oxygen (O) |
| 4.3.2 | Carbon Dioxide (CO2) |
| 4.3.3 | Nitrogen (N) |
| 4.3.4 | Ethylene (C2H4) |
| 4.4 | Temperature |
| 4.4.1 | Lights On |
| 4.4.2 | Lights Off |
| 4.5 | Humidity |
| 4.5.1 | Lights On |
| 4.5.2 | Lights Off |
| 4.6 | Pressure |
| 4.7 | Velocity |
| 5.0 | Purified Water |
| 5.1 | Temperature |
| 5.2 | Oxygen Content (O) |
| 5.3 | Major Nutrients (Salts) |
| 5.3.1 | Nitrogen (N) |
| 5.3.2 | Phosphorus (P) |
| 5.3.3 | Potassium (K) |
| 5.4 | Secondary Nutrients (Salts) |
| 5.4.1 | Calcium (Ca) |
| 5.4.2 | Magnesium (Mg) |
| 5.4.3 | Sulphur (S) |
| 5.5 | Micronutrients (Salts) |
| 5.5.1 | Boron (B) |
| 5.5.2 | Zinc (Zn) |
| 5.5.3 | Copper (Cu) |
| 5.5.4 | Manganese (Mn) |
| 5.5.5 | Iron (Fe) |
| 5.5.6 | Chlorine (Cl) |
| 5.5.7 | Molybdenum (Mo) |
| 5.6 | Vitamin B |
| 5.7 | Hormones |
| 5.7.1 | Inoacetic Acid (IAA) |
| 5.7.2 | Naphthaleneacetic Acid (NAA) |
| 5.7.3 | Cytokinins (CKs) |
| 5.7.4 | Gibberellins (GAs) |
| 5.7.5 | Abscisic Acid (ABA) |

All operations in a production facility will be captured in a Supervisory Control and Data Acquisition (SCADA) system designed to integrate Building Management Systems (BMS), Environmental Monitoring Systems (EMS), Enterprise Resource Planning (ERP) and inventory tracking software. The design of the SCADA system mirrors the facility and process design and is intended to capture data relating to all essential activities in the facility.

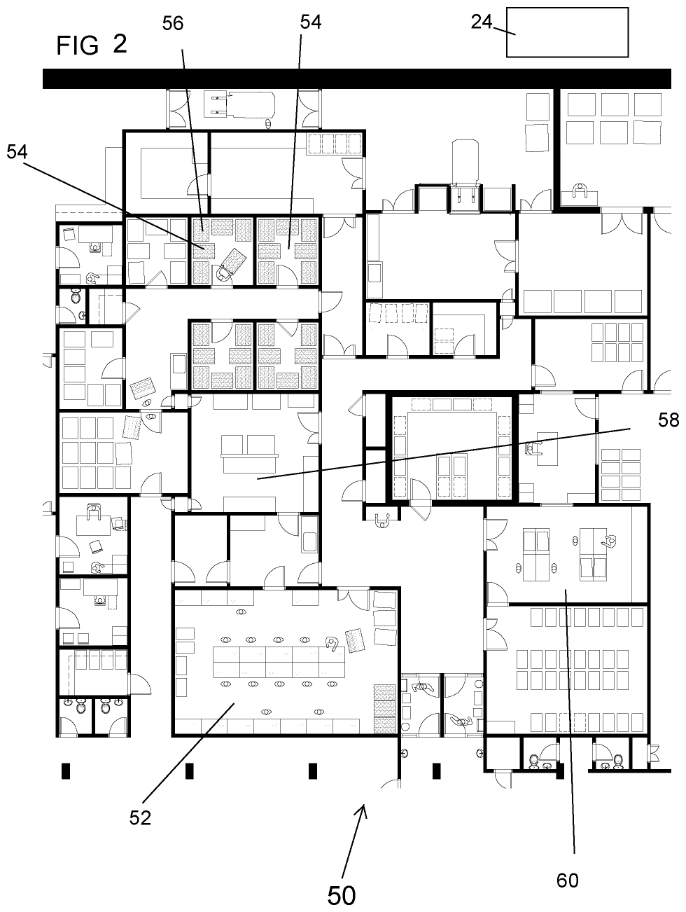

The calibration of the EMS in each growth chamber allows growers to replicate the ‘native’ habitat under which a specific cannabis variety is genetically predisposed to thrive. For the purpose of completeness, block symbols labelled with reference numeral 24 have been added to FIG. 1 and FIG. 2 to generally reference the EMS and BMS which allow the growing environment in that growth chamber to be modified on a day-to day basis to provide the optimal heat, light, water, atmosphere, growing substrate, and nutrients for each stage in the plant's development, from rooted cutting through to mature plant ready for harvest. In the present invention, the cannabis plants remain in the same growth chamber 12 from rooted cuttings to harvest.

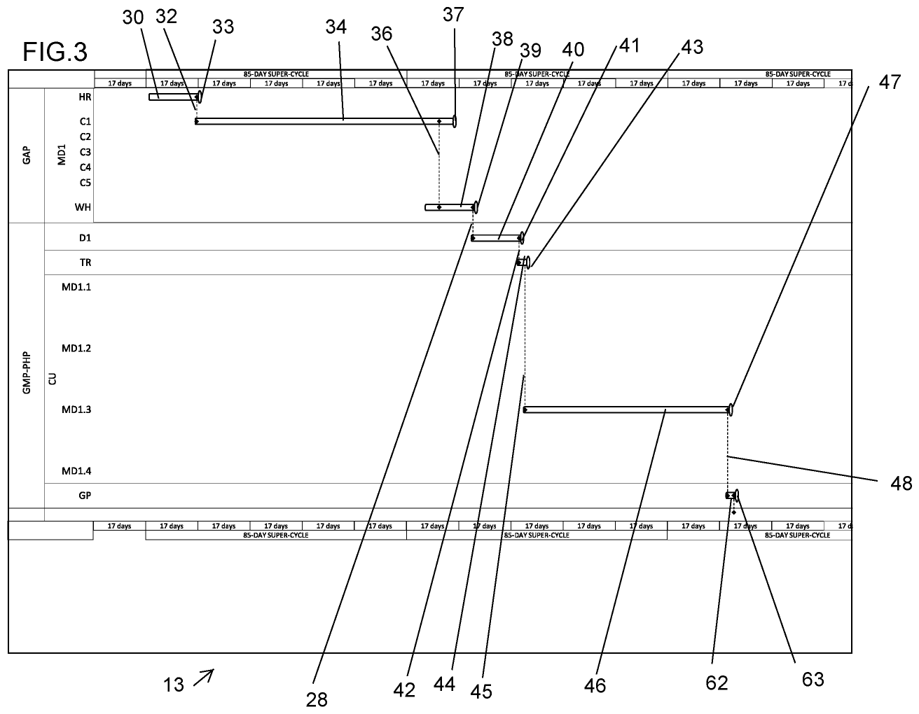

Turning now to FIG. 1 and FIG. 3, the process steps which are completed in the support operations chamber 14 and each of the growth chambers 12 will be discussed in detail. The planting of individual growth chambers 12 within a production module is sequenced and synchronized, so that planting and harvesting activities take place at different times, eliminating the risk of mix-ups and cross-contamination during product transfers. The growth chambers 12A, 12B, 12C, 12D, and 12E are planted sequentially so that when the first growth chamber 12A is ready for harvest, a new batch of starting materials is already assembled in the hardening room 20 to begin the growth cycle all over again with only one ‘down day’ whilst the growth chamber 12A is being sanitized to receive a new planting.

Plants can either be started from seed or by taking cuttings from selected mature plants, known as mother plants. A mother room (not shown) may be provided for cuttings removed from mother plants. One dedicated mother room is sufficient to supply one building module. Cuttings are taken from mother plants to supply growth chambers once per cycle. In the alternative, plant cuttings can be sourced from nurseries or from mother plants located off-site. In any event, the plant cuttings 26 are started in a hardening (rooting) room 20. A hardening (also known as a rooting) step, is identified by reference 30 in FIG. 3. In this step the delicate cuttings will grow roots and become acclimatized to the growth conditions which they will experience in a growing chamber 12. The cycle length of the hardening step 30 is only one MCT, which in the example of a cycle lending itself to a sativa-dominant plant type shown in the present example, is 17 days. Accordingly, only one hardening room 20 is required to supply the five growth chambers 12 in a building module 11. At the end of one hardening cycle 30, the batch of cuttings 26 is transferred to a selected one of the plurality of growth chambers 12, according to a pre-programmed sequence. In the example illustrated in FIG. 3, the transfer step is shown by the dotted line labelled 32, which illustrates the physical movement of the batch of cuttings 26 from the hardening room (identified in production plan 13 shown in FIG. 3 by code “HR”) to the first growth chamber 12A (code “C1”). After the batch of cuttings is removed from the hardening room, a validated cleaning process is executed in the hardening room to prepare for the next batch of cuttings. The cleaning process step is one day long and is indicated in FIG. 3 by the oval symbol labelled with reference numeral 33. In FIG. 1, the growth chamber 12A is planted with new cannabis cuttings 26 that have taken root. Workers will ensure that all hallways making up the path from the hardening room 20 to the growth chamber 12A have been closed off from other areas and cleaned, then the cuttings are moved on transport carts to the growth chamber 12A where they are planted in a selected growth medium. A growing step, (identified in FIG. 3 by reference numeral 34) is five MCT cycles long and the planted cuttings 26 will remain in growth chamber 12A until they are fully grown and ready for harvest.

There is some flexibility in the duration of growing step 34 of the processing cycle. The dotted line 36 indicating the harvest and transfer of the batch of cuttings to the wet hanging room 22 is shown at a position a few days before the end of the full growth period. Even with the best predictive information and environmental controls, a plant will fully mature and become ready for harvest at its own pace. In FIG. 3, the dotted line 36 represents a target zone for when the harvest and transfer step 36 should occur. It will be noted that this line is not shown at the end of the growth cycle 34, to indicate that there is slack in the production plan at this point and indicates the presence of a harvest window. This target zone provides an expansion joint which offers flexibility in the process cycle. If the plants are not quite ready for harvest as of the target zone date, they are allowed to remain in the growth chamber for a few days longer until fully mature and are removed prior to the end of the growth cycle 34. If the plants mature more quickly, they can be harvested and transferred to the wet hanging room 22 to begin the wet hanging stage 38 in advance of the end of the growth cycle 34. After the harvest has been removed from the growth chamber 12A, the growth chamber 12A must be cleaned prior to receiving any new plant material. A cleaning step 37 for the growth chamber (CI) is a one-day step scheduled the day after the completion of the growth cycle 34.

During the wet hanging stage 38 harvested plants will hang in the wet hanging room for 1 MCT cycle (17 days in the example illustrated in FIG. 3) after which they are moved out of the building module 11 for further downstream drying and processing, as will be discussed further below. The wet hanging process step 38 will allow for variance in harvest times. Plants could remain in the wet hanging room 22 until downstream processing facilities are ready to receive them. Shortening the length of the wet hanging step can increase production efficiency generally, but also increases the risk of having no place to store plants in the event of a downstream process or equipment failure.

After a cycle of wet hanging 38 in the wet hanging room 22, the harvested cannabis plants are ready for further processing. Samples are taken for quality control testing to determine whether the plants meet pre-defined Critical Quality Attributes (CQAs) according to the parameters listed in Table 2. The CQAs of the cannabis products are measured in a lab throughout production (in-process) and/or at completion to prepare an attribute profile for the batch of plants. The CQAs are provided with “Q-numbers” so that they can be tracked and correlated against other variables in accordance with the processing plan.

TABLE 2
CANNABIS PLANT - CRITICAL QUALITY ATTRIBUTES

| | | Type of |
|---|---|---|
| Q-Number | Attribute | Attribute |
| 1.0 | Appearance | Quality |
| 1.1 | bud size | Quality |
| 1.2 | bud density | Quality |
| 1.3 | trichomes visible | Quality |
| 1.4 | odour | Quality |
| 1.5 | taste | Quality |
| 2.0 | Identity | Efficacy |
| 2.1 | microscopy | |
| 2.2 | thin layer chromatography | |
| 3.0 | Foreign Material | Quality |
| 4.0 | Fineness | Quality |
| 5.0 | Absence of Pesticides | Safety |
| 6.0 | Microbiological Purity | Safety |
| 6.1 | total aerobic microbial count (TAMC) | |
| 6.2 | total yeast and moulds count (TYMC) | |
| 6.3 | P. aeruginosa, S. aureus and Bile tolerant | |
| | gramneg bacteria | |
| 7.0 | Absence of Heavy Metals | Safety |
| 7.1 | lead max | |
| 7.2 | mercury max. | |
| 7.3 | cadmium max. | |
| 7.4 | arsenic (indicative) - | |
| 7.5 | nickel (indicative) - | |
| 7.6 | zinc (indicative) - | |
| 8.0 | Absence of Aflatoxins | Safety |
| 9.0 | Loss on Drying | Efficacy |
| 10.0 | Assay | Efficacy |
| 10.1 | fingerprint -- -- | Efficacy |
| 10.2 | cannabinoid content -- -- -- -- | Efficacy |
| 10.2.1 | THC | Efficacy |
| 10.2.2 | CBD | Efficacy |
| 10.2.3 | CBN | Efficacy |
| 10.2.4 | CBG | Efficacy |
| 10.3 | major terpenoid content | Efficacy |
| 10.3.1 | terpinolene | Efficacy |
| 10.3.2 | limonene | Efficacy |
| 10.3.3 | pinene | Efficacy |
| 10.3.4 | α-terpineol | Efficacy |
| 10.3.5 | linalool | Efficacy |
| 10.3.6 | myrcene | Efficacy |
| 11.0 | Related Substances | Efficacy |
| 11.1 | cannflavin A & B Content | Efficacy |
| 11.2 | flavonoid content | Efficacy |
| 11.3 | minor terpenoid content | Efficacy |
| 11.3.1 | alpha-phellandrene | Efficacy |
| 11.3.2 | beta-ocimene | Efficacy |
| 11.3.3 | geranyl-acetate | Efficacy |
| 11.3.4 | γ-terpinene | Efficacy |
| 11.3.5 | valencene | Efficacy |
| 11.3.6 | delta-3-carene | Efficacy |
| 11.3.7 | alpha-terpinene | Efficacy |
| 11.3.8 | beta-pinene | Efficacy |
| 11.3.9 | 1,8-cineole | Efficacy |
| 11.3.10 | fenchyl-alcohol | Efficacy |
| 11.3.11 | bisabolol | Efficacy |
| 11.3.12 | camphene | Efficacy |
| 11.3.13 | beta-caryophyllene | Efficacy |
| 11.3.14 | gamma elemene | Efficacy |
| 11.3.15 | caryophyllene-oxide | Efficacy |
| 11.3.16 | alpha-humulene | Efficacy |
| 11.3.17 | guaiol | Efficacy |
| 11.3.18 | γ-eudesmol | Efficacy |
| 12.0 | Ash Content | Quality |

As mentioned above, the cannabis production facility 10 further comprises a GMP processing area 50 which is dedicated to post-harvest processing. As will be discussed with respect to FIG. 2 it can be seen that the GMP processing area 50 comprises a trimming room 52, cannabis drying areas 54 containing drying racks 56, a quality control laboratory 58, a grading and packing room 60, storage, administration and shipping areas.

After quality control testing for the attributes identified in Table 2, the plants are transferred to the pharmaceutical processing area 50 and moved into a drying room 54 and hung on drying racks. In FIG. 3, this transfer step is identified by the dotted line labelled with reference numeral 28. The plants are dried to a pre-determined moisture content during the drying cycle 40.

Further samples are taken at the end of the drying process and again tested in the quality control lab 58 to ensure that no contamination of the plants occurred during drying. The dried plants are then moved in transfer step 42 to a trimming room 52. In a trimming cycle 44 the plants are trimmed and finished flower is placed into curing containers.

The trimming room 52 is a common room, meaning that there is a single trimming room 52 to support all growth chambers. Plant material from every one of the growth chambers 12 will pass through the same trimming room 52 using temporal separation (i.e., based on pre-programmed schedule of activities) to avoid mixing of product materials. The duration of the trimming step 44 for one batch of plant material from one growth cycle in one growth chamber is determined on the basis of the number of growth chambers in operation. One day cleaning cycles must be completed after each batch of plant material is trimmed. The cleaning cycle for the trimming room (code ‘TR’) is shown by reference numeral 43 in FIG. 3.

The trimmed finished flower is transferred (transfer step 45) to a curing room 54, where it will remain for the curing step 46. The curing room 54 is equipped with a plurality of curing bins so as to keep separate the finished flower originating from each individual batch grown in each one of the plurality of growth chambers. The length of the curing step 46 is typically 5 cycles in length but may vary depending upon desired product characteristics and quality. The dried product is then transferred at step 48 to a grading/packing area 60 for a grading/packing cycle 62. After the product has been graded and packed it leaves the process cycle for shipping or warehousing. The grading/packing area 60 is then cleaned during a 1-day cleaning cycle 63 prior to receiving another batch of plant material to repeat the cycle step.

Table 3 summarizes the critical process steps, each of which is related to a physical location in the building module 11 and/or the GMP Pharmaceutical processing area 50 which must be performed to produce a batch of cannabis product.

| P. No. | Process Step |
|---|---|
| 1.0 | Nursery (Tissue Culture Lab) |
| 2.0 | Hardening Room |
| 3.0 | Chamber |
| 4.0 | Wet Hang Room/ |
| 5.0 | In-Process QC (Laboratory) |
| 6.0 | Transfer |
| 7.0 | GMP Drying Room |
| 8.0 | In-Process QC (Laboratory) |
| 9.0 | Trimming |
| 10.0 | Curing (Ageing/Sweating) |
| 11.0 | Grading & Packaging |
| 12.0 | Vault Storage |
| 13.0 | Shipping/Labelling |

Each of the process steps is associated with “P-number” so that the performance of each process step can be recorded for each processing cycle operated. Each process step, as identified by a P-number has associated with it one or more “M-numbers” for each process step and unit operations within. All quality attributes of the cannabis products produced are a function of the materials inputted and the process steps conducted (Q as a function of M*P). Each of the CMAs such as air/gasses, light intensity, etc. must be controlled to within certain upper and lower limits. Critical Process Parameters (CPPs) are the operating ranges within which the Critical Materials Attributes must be maintained. Accordingly, the CPPs are applied to the CMAs at each process step.

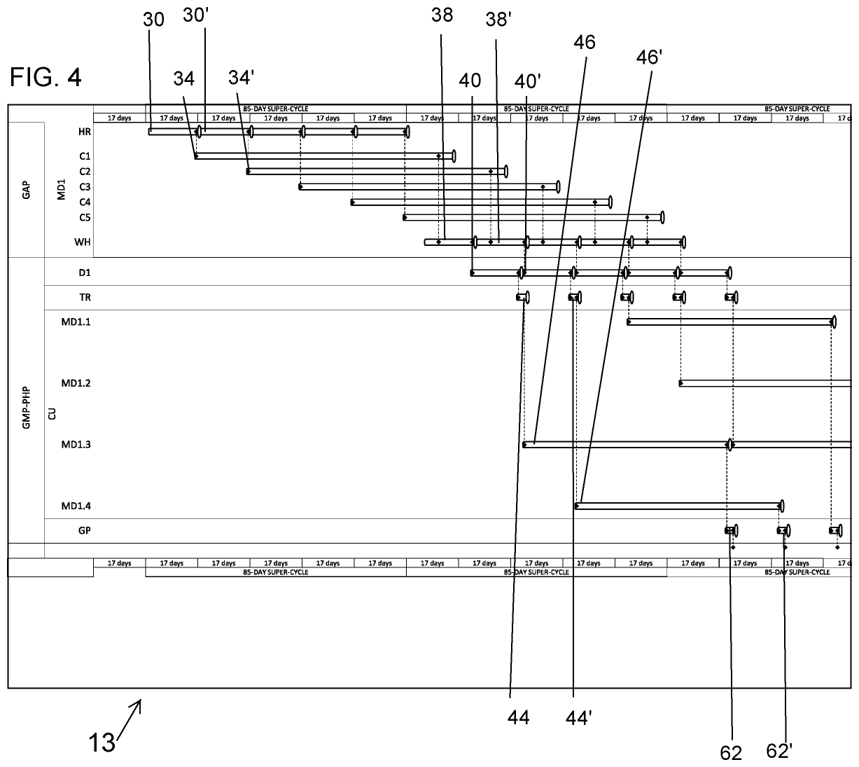

Once the processing steps for an individual batch are visualized and understood, it is easier to visualize the production plan as a synchronized sequence of processing steps which are repeated cyclically. FIG. 4 shows and labels the main process steps for the processing of product from growth chamber 12A as discussed above with reference to FIG. 3: hardening step 30, growth step 34, wet hanging step 38, drying step 40, trimming step 44, curing step 46 and grading/packing step 62. Additionally, FIG. 4 shows the main process steps for batches of plants sequentially planted and hardened, grown, harvested and wet-hung, dried, trimmed, cured, and graded/packed in each of the plurality of growth chambers 12A, 12B, 12C, 12D, and 12E. In order to understand the temporal off-sets between one process and the next for sequentially planted batches, one can compare the corresponding steps for product from growth chamber 12B: hardening step 30′, growth step 34′, wet hanging step 38′, drying step 40′, trimming step 44′, curing step 46′ and grading/packing step 62′. In each instance the corresponding steps are offset from one another by minimum cycle time (MCT), which in the example illustrated is 17 days. It also becomes clear that in addition to temporal separation there is physical separation. Comparing growth step 34 with growth step 34′ shows that a batch of cuttings was transferred from the hardening room to growing chamber 12A (C1) for the first batch, and a second batch of cuttings was transferred from the hardening room to growing chamber 12B (C2) for the second batch.

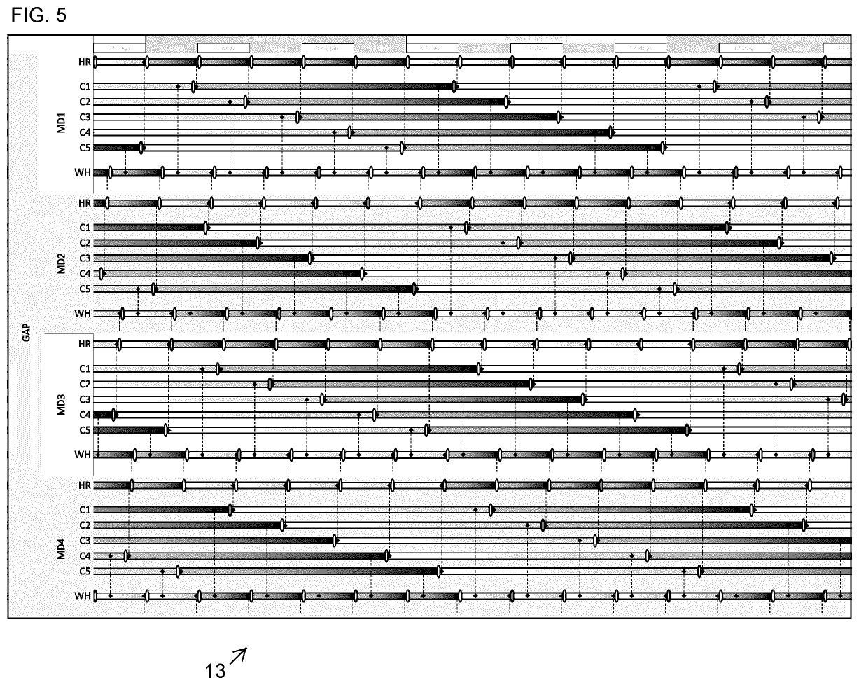

When the main processing steps are run sequentially and cyclically through multiple repetitions, it becomes possible to recognize steady state processing patterns. FIG. 5 is a steady state chart of the hardening, growth, and wet hang processes which occur in the building module 11.

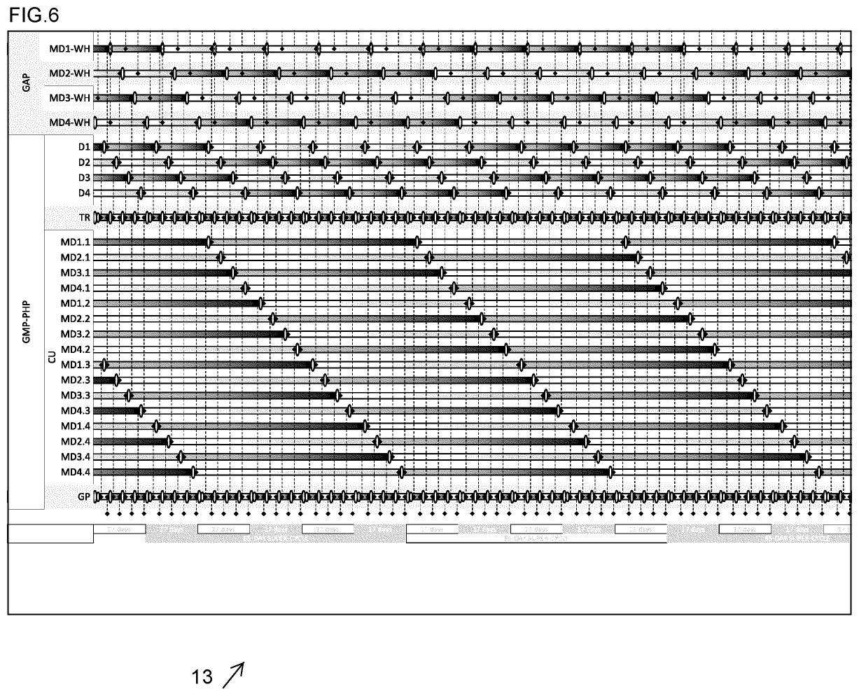

FIG. 6. is a steady state chart of the drying, trimming, curing and grading/packing steps which occur in the GMP pharmaceutical production area 50 To provide additional terms of reference, the wet hanging process steps which occur in the building module 11 are also shown at the top of the chart. FIG. 6 illustrates the fact that cycle lengths in GMP pharmaceutical production area synchronize with cycles predetermined and used in growth process which is carried out in the building module 11. The result can be classed as a System of Concurrent Cyclic Processes (SCCPs). When operating in a steady cyclic state, all material transfers and downstream activities of all modules are harmonized.

Harmonization of all activities to the minimum cycle time provides temporal separation of activities, thus eliminating the risk of human error/accidents whilst operating at approaching 100% efficiency in terms of space usage/room occupation, in other words achieving the maximum attainable return on capital investment. In order to maintain 100% efficiency across the entire system of cyclic production processes, all 5 growth chambers need to be in continuous operation. The production plan 13 incorporating SCCPs operates at 100% of available capacity within each process step, with the only exception being a harvest window in the growth step 34 and created by the target zone for when the harvest and transfer step 36 should occur, as discussed above. The harvest window acts as an “expansion joint” within a rigid fixed process allowing for variation within a variable horticultural process (the duration of the window subject to a grower's familiarity with a particular phenotype and risk tolerance). The efficiency of the cyclic processes occurs because the timing/duration of all downstream activities is sequenced based on the minimum cycle length or portions/multiples thereof. Common rooms, where the unit operation is based on an activity as opposed to length of time such as the trimming room, must process an entire batch within a “sub-cycle” with a duration of 1 cycle divided by the number of modules in operation’ (where the cleaning process between batches is included in the sub-cycle). Additionally, modules with different minimum cycle lengths can be operated in the same facility with minor “peaks and troughs” in terms of common room staffing whilst maintaining temporal separation between batches, an example would be the 13-day cycle process suited to most indica type cannabis plants and the 17-day cycle (as per example) suited to most sativa type plants.

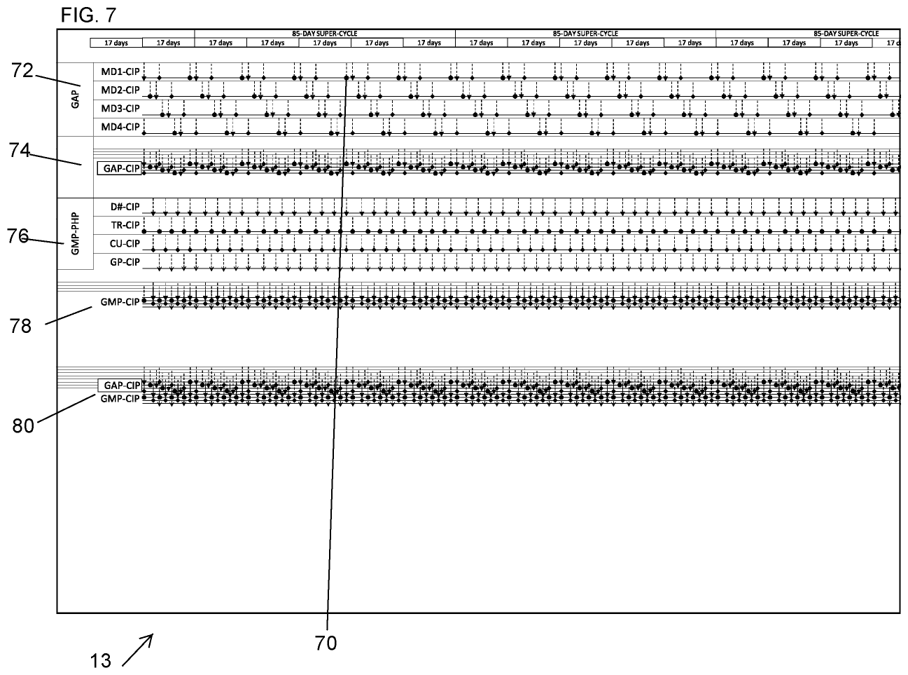

FIG. 7 shows all of the cleaning steps for all sterile areas of the production facility plotted as a steady state chart. Each individual black dot 70 corresponds to an oval symbol appearing on the production plan drawings previously discussed and represents a discrete cleaning task. Reference 72 refers to the cleaning tasks which occur in the hardening room 20 and in each of the growth chambers 12A, 12B, 12C, 12D, and 12E. Reference 74 shows a stacked view of all of the cleaning activities which occur in the growth chambers and the rooms of the supporting activity area 14. Reference 76 shows each of the cleaning tasks which occur in the drying rooms 54, the trimming room 52, the quality control lab 58, the curing areas, and the grading/packing area 60. Reference 78 shows a stacked view of all cleaning tasks which occur in the GMP pharmaceutical processing area 50. Finally, reference 80 is a stacked view of all cleaning tasks in the entire processing facility 10. Although there are many sequenced cleaning tasks, one can tell by comparing the stacked views 74, 78, and 80 that the dots 70 do not overlap, meaning that there are no points in the production plan 13 where conflicts occur with regard to cleaning tasks that must be performed, but rooms have not yet been emptied to allow for cleaning. Additionally, the portion of the production plan shown in FIG. 7 can be used to calculate manpower and materials requirements to enable cleaning tasks which must be completed at any given time.

Enterprise Resource Planning (ERP) software integrates the progress of activities within the facility with bills of materials associated with each batch of products, tracking the materials used in its production and associated costs. When stocks of materials fall below a specific threshold, the system will generate a purchase order so that a new supply of materials arrives at the facility well before the previous one is fully depleted. The integration of ERP software is possible due to the regular pattern of activities dictated by the production plan, and the resulting production facility becomes more than just a continuous manufacturing facility. It becomes a smart facility. The integration of ERP does not change the manufacturing process itself, but allows for planning well into the future.

As a result of the regular cadence of activities controlled according to the production plan, a production facility according to the present invention approaches continuous manufacturing, a significantly more efficient method of production compared to batch manufacturing, the current standard in the cannabis industry.

The modular design of the production facility 10 accommodates ease of expansion of facility capacity and technology transfer without disrupting existing operations. Individual building modules 11, can be built in different sizes, depending on building limitations and production objectives. Regardless of their size, each building module 11, will comprise five growth chambers 12 and one support operations chamber 14 dedicated to activities in support of the growth chambers 12, as discussed above. Additional building modules 11 can be added to increase production capacity while using a single GMP processing area 50, thereby maximizing the use of laboratory and support facilities. Design and operational specifications corresponding to individual production modules 11 change in parallel with their scale, to ensure that all systems work in harmony and all modules produce uniform data sets, regardless of their size. For optimal production efficiency, a processing facility could operate up to 4 building modules serviced by a single GMP processing area.

The scalable nature of the production facility and the complete integration of the facility infrastructure with the digital infrastructure create an opportunity for large-scale data collection and research. The digital infrastructure has been designed to ensure that sensors used to support process controls capture quantitative and qualitative data about the cannabis plants at various stages of the manufacturing process. The continuous flow of uniform, production-related information from each production module in every production facility enables the generation of valuable data from commercial harvests without the need for separate and costly R&D facilities. This collected data is used to identify correlations between variations in environmental factors and outcomes such as the plants' morphological traits or the production, accumulation or degradation of specific chemical compounds within the plants. Statistically significant data trends will inform decisions about the calibration of future manufacturing systems and processes to achieve specific product characteristics, such as larger and denser flowers, curated terpene profiles, or increased content of rare cannabinoids. When operating at scale, the circular data flow from all monitored infrastructure and processing steps in the ecosystem has the potential to significantly accelerate production innovation and research into cannabis and its uses in medicine and daily life.

The production plan enables the generation of detailed and accurate production plan prototypes for operation of production facilities. At the earliest conceptual stage of a new project design the production plan is capable of calculating production capacity, both facility and staffing requirements, and importantly a cost per unit of production (i.e., the industry standard “cost per gram”). Therefore, determining the feasibility of a proposed project is possible without the considerable effort of undertaking the numerous stages of conventional building and manufacturing design with the associated costs of external consultants.

The production plan can accommodate any type of cannabis (or plant) and associated process parameters can be adjusted manually or updated from a catalogue of “recipes” to suit desired product characteristics. In jurisdictions where the facility must demonstrate GMP compliance for licensing purposes, the production plan can save time and resources which would otherwise have to be spent on validating a proposed new process. In jurisdictions where cannabis production is permitted without requirement of compliance with medicinal regulations, manufacturers have greater flexibility to change individual process parameters in line with their grower's preference. Likewise, the production plan makes it possible to operate several facilities utilising a single individual's skillset, or “recipe”.

The five-chamber design of the module itself lends itself to the design of experiments, which in turn inform future production plans. As an example, building module can determine the optimum combination of three variables in as little as three super-cycles (fifteen minimum cycle lengths) through comparing the Certificate of Analyses from fifteen individual harvests and then constructing a three-dimensional model from the result of a myriad of combinations of variables with respect to any particular quality characteristic. This is achieved by translating three process parameters into an x, y and z axis and adjusting the variables per chamber per cycle as follows:

SUPER-CYCLE NO. 1

| Chamber | X-Axis | Y-Axis | Z-Axis | Positioning |
|---|---|---|---|---|
| 1 | −5X | −5Y | −5Z | lower plane, bottom left |
| 2 | +5X | −5Y | −5Z | lower plane, bottom right |
| 3 | +5X | +5Y | −5Z | lower plane, upper right |
| 4 | −5X | +5Y | −5Z | Lower plane, upper left |
| 5 | 0X | 0Y | −5Z | Lower plane, centre |

SUPER-CYCLE NO. 2

| Chamber | X-Axis | Y-Axis | Z-Axis | Positioning |
|---|---|---|---|---|
| 1 | −5X | −5Y | −5Z | lower plane, bottom left |
| 2 | +5X | −5Y | −5Z | lower plane, bottom right |
| 3 | +5X | +5Y | −5Z | lower plane, upper right |
| 4 | −5X | +5Y | −5Z | Lower plane, upper left |
| 5 | 0X | 0Y | −5Z | Lower plane, centre |

SUPER-CYCLE NO. 3

| Chamber | X-Axis | Y-Axis | Z-Axis | Positioning |
|---|---|---|---|---|
| 1 | −5X | −5Y | −5Z | lower plane, bottom left |
| 2 | +5X | −5Y | −5Z | lower plane, bottom right |
| 3 | +5X | +5Y | −5Z | lower plane, upper right |
| 4 | −5X | +5Y | −5Z | Lower plane, upper left |
| 5 | 0X | 0Y | −5Z | Lower plane, centre |

From the above results a 3-D model can be extrapolated which illustrates varying effects on quality attributes (e.g., linalool content) and the highest reading with respect to said attribute and associated parameter settings clearly indicated. This information can inform future calibration of BMS and EMS, process improvements, and identify areas for further study.

The modular design with variable sizes/proportions is based on super-skid philosophy, with tech-transfer in mind, to suit any existing open plan building as well as new sites/developments. Any number of modules can be deployed with physical separation provided by the building fabric and controlled access of materials and personnel into and within the module. For example, once an individual has entered a chamber via activation of a fob swipe (or similar technology), the building management system (BMS) records the movement of the individual into that chamber and will not allow access to a second chamber without the individual having been recorded as existing and re-entering the module through personnel control points, with associated gown down/gown up regimes and security procedures.

The production plan and cost analysis can be provided based on the following six variables. (1) What is the desired canopy size per chamber? The canopy refers to the size of the vegetative canopy produced by plants as they grow and mature. Inputting a selected value for the canopy space determines the projected batch size of product which can be produced, the product throughput and the projected energy consumption for the proposed processing facility.

(2) What type of plant will be grown/what is the proposed time to harvest? Inputting a value for this variable provides the projected yield per square metre and the environmental/lighting intensity specification for determining projected batch size/throughput and energy consumption. Additionally, the type of genetic starting material dictates the flowering time required or duration of the chamber process step which then determines the minimum-cycle-length and timing of all downstream activities/process steps.

(3) How many building modules are proposed at the facility? Inputting a value for this variable provides the total throughput/production capacity as well as determining the sub-cycle-time allowed for operations in combined rooms (e.g., trimming) where the total throughput of the facility must be processed within a single minimum-cycle-time.

(4) How long is the minimum ageing/curing duration? Inputting a value for this variable determines the number of curing bins and associated floorspace required in the curing room. The number of bins required is equal to the number of cycles from which the curing materials are generated, to allow for projected throughput.

(5) How long is desired for a harvest window allowing for crop-to-crop variations? Inputting a value for this variable determines the total manufacturing process duration from cutting to finished packaged product; the longer the harvest window the less efficient the chamber process step and wet hang steps are. Allowing a longer harvest window means that cannabis plant material will have less overall time for the slow-drying process (comprising wet hanging and GMP drying) prior to trimming. Allowing a longer harvest window also provides a greater tolerance in the production plan for inconsistencies in growing times or processing failures downstream. A harvest window of only 3 days would provide for maximum efficiency with minimal risk mitigation.

(6) What are the input costs for electricity; input materials such as growing media, fertilizers, water, nursery cuttings, etc.; and, staffing costs in terms of hourly wages, payroll deductions, benefits, etc? These final inputs, when applied to the calculated areas, staffing requirements (as based on multiples of repetitive and predictable unit operations), and consumption of materials and utilities as all can be readily calculated based on the SCCPs production plan to generate accurate projections of production costs, or cost per unit (cost per gram). Capital expenditures can also be accurately calculated based on a modular design of precisely sized rooms/components and areas.

From the options selected above, a facility design and production plan can be produced, allowing various options/alternatives to be evaluated from the feasibility/concept stage through to on-going operations. The predictable nature of SCCPs also make it possible to apply ERP software to further improve efficiency of operations. The key lies in the original design of the production module comprising 5 growth chambers and the timing of successive plantings/harvests. In any manufacturing operation, the efficiency is limited by the longest process step in so much as there will inevitably be a bottleneck. In the case of chemical manufacturing this is usually the duration of the process step performed by a specific piece of equipment, and the bottleneck can be eased by the addition of additional equipment. In the case of the production facility according to the present invention, the plant growing time is the longest process step, and in order to ease the processing bottleneck, the number of growth chambers are increased, thereby reducing the process length. In essence, the architecture dictates the minimum cycle time which informs the SCCPs and the ability to accurately plan and predict future activities. As discussed above, the minimum-cycle-time (MCT) is determined as ⅕^{th} of the time required for a single growth chamber from planting until harvest (with some allowance in a harvest window). This MCT is then applied to all downstream activities such as GMP drying which is one full cycle length, or the curing time being a multiple of cycles. Finally, the relationship to the trimming process step and the grading and packaging is critical to mitigating risk of cross contamination/product mix-up as when these are aligned (due to the fact the curing time is a function of cycle time) then all materials in the GMP processing area in operation at any given time derive from one module. Therefore, the modules when scheduled as described above not only offer greater efficiency and production capacity but also inherent compliance with cGMP regulations and best practice through simplifying quality risk management (QRM) and quality management systems (QMS).

Coding used in the production plan 13 appears in each of FIG. 3 through FIG. 7 to identify the principal rooms in the building module 11 of the production facility. A correspondence key for the room codes in the production plan follows, and a Parts List follows on the next subsequent page.

| | | Parts |
|---|---|---|
| Production plan code | Room name | List # |
| HR | hardening room | 20 |
| C1 | growth chamber 1 | 12A |
| C2 | growth chamber 2 | 12B |
| C3 | growth chamber 3 | 12C |
| C4 | growth chamber 4 | 12D |
| C4 | growth chamber 5 | 12E |
| WH | wet hang room | 22 |
| DR | drying room | 54 |
| TR | trimming room | 52 |
| CU MD1.1 | curing room for product from C1 | |
| CU MD1.2 | curing room for product from C2 | |
| CU MD1.3 | curing room for product from C3 | |
| CU MD1.4 | curing room for product from C4 | |
| CU MD1.5 | curing room for product from C5 | |
| GP | grading/packing room | 60 |

PARTS LIST

| Reference | |
|---|---|
| Number | Part |
| 10 | Production facility |
| 11 | Building module |
| 12 | Plurality of growth chambers |
| 13 | Production plan |
| 14 | Support operations chamber |
| 16 | Gown-up room |
| 17 | Materials receiving area |
| 18 | Gown-down room |
| 20 | Hardening/rooting room |
| 22 | Wet hanging room |
| 24 | Environmental equipment and BMS |
| 26 | Plant cuttings |
| 28 | Transfer step to drying room |
| 30 | Hardening step |
| 30′ | Hardening step for cutting going to chamber 12B |
| 32 | Transfer step to growth chamber |
| 33 | Cleaning step-hardening room |
| 34 | Growing step |
| 34′ | Growing step in chamber 12B |
| 36 | harvest and transfer to wet hanging room |
| 37 | Cleaning step-growth chamber |
| 38 | Wet hanging step |
| 38′ | Wet hanging step for plants from chamber 12B |
| 40 | Drying cycle |
| 40′ | Drying cycle for plants from chamber 12B |
| 42 | Transfer step to trimming room |
| 43 | Cleaning step-trimming room |
| 44 | Trimming step |
| 44′ | Trimming step for plants from chamber 12B |
| 45 | Transfer step - finished flower to curing room |
| 46 | Curing step |
| 46′ | Curing step for plants from chamber 12B |
| 48 | Transfer step - to grading and packing room |
| 50 | GMP processing area |
| 52 | Trimming room |
| 54 | Cannabis drying areas |
| 56 | Drying bins |
| 58 | Quality control lab |
| 60 | Grading and packing room |
| 62 | Grading/packing step |
| 62′ | Grading/packing step |
| 63 | Cleaning step grading/packing area |
| 70 | Representation of clean step |
| 72 | cleaning task in the hardening room 20 |
| 74 | Stacked view of cleaning tasks in growth chambers and |
| | supporting activity area |
| 76 | Cleaning tasks in the support operations chamber |
| 78 | Stacked view of all Cleaning tasks in GMP processing area |
| 80 | Stacked view of all Cleaning tasks in processing facility |

We claim:

1. A production plan for producing cannabis in a facility comprising a building module comprising five growth chambers of substantially equal size; each said growth chamber being fully equipped to provide an optimal growing environment having all critical material attributes (CMAs) necessary to support the natural lifecycle of the cannabis plant; a building management system (BMS) and environmental management system (EMS) comprising a system of sensors calibrated for monitoring critical process steps as they relate to the critical material attributers (CMAs) and support operations chamber; and a GMP processing area; said production plan comprising:
a system of concurrent cyclic processes (SCCPs) by which the following cannabis processing steps are carried out: hardening; growth, wet hanging, in-process quality control, transfer to GMP drying; in-process quality control; trimming; curing, and grading/packing.

2. The production plan of claim 1, wherein the SCCPs is based upon a minimum cycle time (MCT) defined as MCT=FP/5, where FP is a flowering period for a selected cannabis species, and 5 is the number of growth chambers in which the cannabis species will be grown.

3. The production plan of claim 2, wherein:
the cycle length of the hardening step is 1 MCT;
the cycle length of the growing step is 5 MCT;
the cycle length of the wet hanging step is 1 MCT;
the cycle length of the GMP drying step is 1 MCT;
the cycle length of the curing step is at least 3 MCT; and,
the grading/packing cycle is 1 MCT.

4. The production plan of claim 3, further comprising a plurality of cleaning sub-cycles interleaved between each of the processing steps.

5. The production plan of claim 4, wherein the cycle length of each of the cleaning sub-cycles is one day.

6. The production plan of claim 5, wherein the trimming step is performed in a common room and has a unit of operation based upon the performance of trimming activity, such that each batch of cannabis is trimmed within a sub-cycle having a duration of 1 MCT divided by 5, where 5 is the number of growth chambers being operated.

7. A cannabis production facility comprising:
a building module comprising five growth chambers of substantially equal size; each said growth chamber being fully equipped to provide an optimal growing environment having all critical material attributes (CMAs) necessary to support the natural lifecycle of the cannabis plant; a building management system (BMS) and environmental management system (EMS) comprising a system of sensors calibrated for monitoring critical process steps as they relate to the critical material attributers (CMAs); and a support operations chamber;
a GMP processing area dedicated to post-harvest processing; and,
a production plan, being a system of concurrent cyclic processes (SCCPs) by which the following cannabis processing steps are carried out: hardening; growth, wet hanging, in-process quality control, transfer to GMP drying; in-process quality control; trimming; curing, and grading/packing.

8. The cannabis production facility according to claim 7, wherein the support operations chamber comprises: a gown-up area; a materials receiving area; a hardening room; a wet hanging room; and, a gown-down area.

9. The cannabis production facility according to claim 1, wherein the GMP processing area comprises: a drying area; a quality control laboratory; a trimming room; a curing area; and a grading and packing area.

10. A method for designing a cannabis production facility for assessing the feasibility thereof at a concept stage comprises the steps of:
providing a production plan for producing cannabis in a facility comprising a building module comprising five growth chambers of substantially equal size; each said growth chamber being fully equipped to provide an optimal growing environment having all critical material attributes (CMAs) necessary to support the natural lifecycle of the cannabis plant; a building management system (BMS) and environmental management system (EMS) comprising a system of sensors calibrated for monitoring critical process steps as they relate to the critical material attributers (CMAs) and support operations chamber; and a GMP processing area; said production plan comprising a system of concurrent cyclic processes (SCCPs) by which the following cannabis processing steps are carried out: hardening; growth, wet hanging, in-process quality control, transfer to GMP drying; in-process quality control; trimming; curing, and grading/packing;
determining a cannabis canopy size value per growth chamber and inputting said cannabis canopy size value into the production plan;
determining a flowering period value for a selected cannabis species and inputting said flowering period value into the production plan;
determining a desired number of building modules each of which comprises five growth chambers and inputting the desired number of building modules into the production plan;
determining a desired minimum curing duration and inputting a value for minimum curing duration into the production plan;
determining a desired harvest window value and inputting the desired harvest window value into the production plan; and,
determining a value for anticipated input costs and inputting the value for anticipated input costs into the production plan.
